(19) United States
(12) Patent Application Publication
(10) Pub. No.: US 2026/0015576 A1
(43) Pub. Date: Jan. 15, 2026

(54) SYSTEMS AND METHODS OF ADAPTIVE LABORATORY EVOLUTION AND ANALYTICS USING MICROFLUIDIC DROPLETS AND BIOSENSORS

(71) Applicant: UCHICAGO ARGONNE, LLC, Chicago, IL (US)

(72) Inventors: Gyorgy Babnigg, Countryside, IL (US); Jessica L. Johnson, Plainfield, IL (US); Philip D. Laible, Downers Grove, IL (US); Rosemarie Wilton, Elmhurst, IL (US); Chase Akins, Marengo, IL (US)

(21) Appl. No.: 19/205,335

(22) Filed: May 12, 2025

Related U.S. Application Data
(60) Provisional application No. 63/670,393, filed on Jul. 12, 2024.

(51) Int. Cl. C12N 5/00; C12M 1/00; C12M 1/12; C12M 3/06

(52) U.S. Cl. CPC C12N 5/0012; C12M 23/16; C12M 25/16; C12M 41/14

(57) ABSTRACT

A droplet adaptive laboratory evolution (dALE) method for generating or obtaining cells adapted to replicate in a selected environment includes creating a plurality of droplets from a first suspension of cells in which each droplet contains one or more cells. The method also includes incubating the plurality of droplets, breaking the droplets, resuspending the cells, re-encapsulating cells from the broken droplets, and repeating this process at least twice, or at least four times. A system adaptable to perform the dALE method for identifying cells adapted to replicate in a selected environment includes a droplet producing device, an incubator, a droplet breaking apparatus, and a cell culture apparatus.

### CROSS REFERENCE TO RELATED APPLICATION

The benefit of priority to U.S. Provisional Application No. 63/670,393 filed Jul. 12, 2024, is hereby claimed and the disclosure is incorporated herein by reference in its entirety.

### STATEMENT REGARDING FEDERALLY SPONSORED RESEARCH OR DEVELOPMENT

This invention was made with government support under DE-ACO2-06CH1137 awarded by the Department of Energy. The government has certain rights in this invention.

### FIELD OF THE DISCLOSURE

The present disclosure relates to methods and systems for growth and identification of cell strains which rapidly multiply, thrive, and exhibit tolerance in selected environments. The methods and systems find particular use in research settings where the initial candidates for viable cells multiply poorly in a selected medium. Thus, there is a need in the art for improved methods of evolving cells for their ability to replicate and thrive under various conditions.

### BACKGROUND

Identification of cell lines capable of thriving in a given environment (e.g., medium and temperature range) is traditionally accomplished by beginning with a large volume of medium in which cells are suspended. The suspension with a mixture of cells is divided into flasks, vials, or wells of cell culture plates. After incubation and reaching saturation, cultures aliquots are diluted and transferred into new culture vessels (passaging). The procedure is repeated for months to produce evolved candidates, due to spontaneous mutations, with rapid multiplication in the given environment. At the end the cultures are spread onto agar plates and colonies picked and isolates characterized, and top performers selected, those that both thrive in a given environment and produce a desired biological product. The characterization can be carried out using traditional analytical methods (e.g., HPLC, LC-MS) or using biosensors with either a fluorescent protein (FP) or an enzyme reporter. Current methods take months to evolve the hardiest cells due to the fact that poor performers can feed on byproducts produced by the top performers (cross-feeding).

The results from cells that are tested in a selected medium gives results for the milieux or mixture of cells. There may be a suppression or amplification effect from a particular group of cells which are cultured together. This may not allow for the hardiest or best adapted cells for the given media to be identified, in turn possibly preventing optimization of the cell selection and, in some cases, production of targeted bioproducts. Thus, there is a need in the art for improved methods for evolving cells for their ability to replicate and thrive under various conditions.

### SUMMARY OF THE DISCLOSURE

In some implementations, a method is provided for generating and/or selecting cells that are adapted to replicate in a selected environment. The method includes the steps of encapsulating cells from a first suspension of cells in a first cell culture medium into a plurality of droplets, each droplet comprising one or more cells from the first suspension of cells; incubating the plurality of droplets in an incubating environment; breaking the plurality of droplets to release cells; resuspending a subset of the released cells in a second cell culture medium to form a second suspension of cells and re-encapsulating cells to form a new plurality of droplets. In the method, the steps of incubating, breaking, and re-encapsulating are repeated so as to identify cells adapted to replicate in the selected environment. The selected environment includes one or more of the first cell culture medium, the second cell culture medium, and the incubating environment.

The following features may be combined in the method in any reasonable manner. The incubating environment includes any one or more of: percent oxygen (O₂), relative humidity, temperature, and agitation. The first and second culture medium includes any one or more of a feed-stock; a toxin; an inhibitor; a salt; a biological product or byproduct; a media component; and cell debris or waste. The incubating, breaking, resuspending, and re-encapsulating steps are repeated at least 4 times. The incubating, breaking, resuspending, and re-encapsulating steps are repeated at least 2 times in a time period of seven days. Incubating the plurality of droplets is done over a period of time equal to at least 12 hours. The method also includes collecting the identified cells that are adapted to replicate in the selected environment. Determining an amount of a desired product or analyte produced by the cells is also part of the method. The method further includes introducing a biosensor to the plurality of droplets that emits a signal when the desired product or analyte is present; and sorting the plurality of droplets based upon a presence or an absence of the biosensor signal in the droplet. In the method, when introducing a biosensor to the plurality of droplets, the step of introducing the biosensor includes any of injecting the biosensor into each droplet of the plurality of droplets using a pico-injection method; co-encapsulation of the biosensor with each cell in each droplet of the plurality of droplets; and merging droplets containing the biosensor with a droplet of the plurality of droplets. In the method, sorting the droplets includes using fluorescence-activated droplet sorting. The biosensor includes a cell-based biosensor in some implementations. Each droplet of the plurality of droplets has a volume ranging from 10 picoliters to about 1000 nanoliters. Each droplet of the plurality of droplets has a diameter ranging from 5 microns to about 2000 microns. The initial suspension of cells includes any of a genetically heterogenous or inhomogeneous cell population; a genetically homogenous cell population; natural cells; engineered cells; and a heterogeneous cell population generated by library approaches. The method includes evaluating the cells adapted to replicate in the selected environment based on ability to produce a desired product in some implementations.

In some implementations, a system for identifying cells adapted to replicate in a selected environment is provided. The selected environment includes an incubating environment, and/or one or more of a first cell culture medium and a second cell culture medium. The system includes a droplet producing device, an incubator, a droplet breaking apparatus, and a cell culture apparatus. The droplet producing device is configured to accept a suspension of cells in the first or second cell culture medium and to create droplets. Each droplet made by the droplet producing devices includes one or more cells from the suspension of cells. The incubator is configured to allow replication of cells within each droplet while in the incubating environment. The droplet breaking apparatus is adapted to accept droplets from the incubator and to yield cells in suspension. The cell culture apparatus is adapted to accept survivor cells in suspension from the droplet breaking apparatus. The cell culture apparatus is also configured to create cell isolates.

The disclosure thus provides a method for generating and/or selecting cells adapted to replicate in a selected environment, the method comprising steps of: encapsulating cells from a first suspension of cells in a first cell culture medium into a plurality of droplets, each droplet comprising one or more cells from the first suspension of cells; incubating the plurality of droplets in an incubating environment; breaking the plurality of droplets to release cells; resuspending a subset of the released cells in a second cell culture medium to form a second suspension of cells; and re-encapsulating cells to form a new plurality of droplets; wherein the incubating, breaking, resuspending, and re-encapsulating steps are repeated to identify cells adapted to replicate in the selected environment. In some aspects, the selected environment comprises a first cell culture medium, a second cell culture medium, and an incubating environment. In some aspects, the incubating environment includes any one or more of: percent oxygen (O₂), relative humidity, temperature, and agitation. In some aspects, the culture medium includes any one or more of: a feed-stock composition; a toxin; an inhibitor; a salt; selected or target ionic strength; a selected or target osmolarity, which can be high or low; a selected or target pH, a biological product or byproduct; a media component; and cell debris/waste.

In some aspects, the incubating, breaking, and re-encapsulating steps are repeated at least 4 times. In some aspects, the incubating, breaking, resuspending, and re-encapsulating steps are repeated at least 2 times in a time period of seven days. In some aspects, incubating the plurality of droplets is done over a period of time equal to at least 12 hours.

In some aspects, the method further comprises collecting the identified cells that are adapted to replicate in the selected environment. In some aspects, the method further comprises determining an amount of a desired product or analyte produced by the cells.

In some aspects, the method further comprises introducing a biosensor that emits a signal when the desired product or analyte is present to the plurality of droplets; and sorting the plurality of droplets based upon a presence or an absence of the biosensor signal in the droplet. In some aspects, introducing the biosensor comprises any of injecting the biosensor into each droplet of the plurality of droplets using a pico-injection method; co-encapsulation of the biosensor with each cell in each droplet of the plurality of droplets; and merging droplets containing the biosensor with a droplet of the plurality of droplets. In some aspects, sorting the droplets comprises using fluorescence-activated droplet sorting. In some aspects, the biosensor comprises a cell-based biosensor. In some aspects, each droplet of the plurality of droplets has a volume ranging from 10 picoliters to about 1000 nanoliters. In some aspects, each droplet of the plurality of droplets has a diameter ranging from 5 microns to about 2000 microns. In some aspects, the initial suspension of cells includes any one of a genetically heterogenous or inhomogeneous cell population; a genetically homogenous cell population; natural cells; engineered cells; and a heterogeneous cell population generated by library approaches. In some aspects, the method further comprises evaluating the cells adapted to replicate in the selected environment based on ability to produce a desired biological product.

The disclosure also provides a system for identifying cells adapted to replicate in a selected environment. The selected environment can include an incubating environment, and/or one or more of a first cell culture medium and a second cell culture medium. The system includes a droplet producing device configured to accept a suspension of cells in the first or second cell culture medium and create droplets, wherein each droplet comprises one or more cells from the suspension of cells; an incubator configured to allow replication of cells within each droplet while in the selected environment; a droplet breaking apparatus adapted to accept droplets from the incubator and yield cells in suspension; and a cell culture apparatus adapted to accept survivor cells in suspension from the droplet breaking apparatus, the cell culture apparatus configured to create cell isolates.

In some aspects, the droplet producing device is a two-stream flow-focusing microfluidic device. In some aspects, the two-stream flow-focusing microfluidic device is further configured to accept the cells in suspension created by the droplet breaking apparatus.

In some aspects, the system further comprises a cell dilution apparatus adapted to accept the cells in suspension created by the droplet breaking apparatus, wherein the cell dilution apparatus is configured to provide the droplet producing device with a subsequent cell suspension.

In some aspects, the incubator is configured to allow replication of cells for at least 12 hours.

In some aspects, the system further includes an apparatus for determining an amount of desired product produced by each cell isolate. In some aspects, the system further includes a biosensor introducing apparatus adapted to accept the droplets from the droplet producing device and insert a biosensor into the droplets. In some aspects, the system further includes a sorting apparatus adapted to accept droplets, wherein each droplet contains a cell and a biosensor, to create a collection of selected droplets.

The disclosure also provides a method for identifying cells adapted to replicate in a selected environment, the method comprising use of the system as disclosed herein.

The following features may be combined in any reasonable manner into the system described herein. The droplet producing device is a two-stream flow-focusing microfluidic device. In systems with a two-stream flow-focusing microfluidic device, that device is also configured to accept the cells in suspension created by the droplet breaking apparatus. The system includes a cell dilution apparatus adapted to accept the cells in suspension created by the droplet breaking apparatus, in which the cell dilution apparatus is configured to provide the droplet producing device with a subsequent cell suspension. The incubator is configured to allow replication of cells for at least 12 hours. The system also includes an apparatus for determining an amount of desired product produced by each cell isolate in some implementations. A biosensor introducing apparatus adapted to accept the droplets from the droplet producing device and insert a biosensor into the droplets is included in the system in some implementations. Additionally, a sorting apparatus adapted to accept droplets is part of the system, and each droplet contains a cell and a biosensor, to create a collection of selected droplets.

In some implementations, a method for identifying cells adapted to replicate in a selected environment uses the system described hereinabove.

### BRIEF DESCRIPTION OF THE DRAWINGS

The figures described below depict various aspects of the apparatus, systems, and methods disclosed therein. It should be understood that each figure depicts an example of a particular aspect of the disclosed apparatus, systems, and methods, and that each of the figures is intended to accord with a possible example thereof. Further, wherever possible, the following description refers to the reference numerals included in the following figures, in which features depicted in multiple figures are designated with consistent reference numerals.

There are shown in the drawing arrangements which are disclosed or discussed herein, it being understood, however, that the examples of the disclosure are not limited to the precise arrangements and instrumentalities shown, wherein:

FIG. 1 is a schematic of a system that uses dALE to generate cells which exhibit hardiness and significant growth, or cell multiplication, in selected media;

FIG. 2 is a schematic of a system for encapsulation of a cell in a droplet of medium;

FIG. 3A shows a representative droplet containing a single cell in a certain medium, at an initial time shortly after formation of the droplet;

FIG. 3B shows a representative droplet after a day of incubation;

FIG. 3C shows a representative droplet after two days of incubation;

FIG. 4A shows an example method where, after dALE, product titers are measured directly in the droplets via biosensors and the signal is used to select top performers via droplet sorting before final isolate characterization;

FIG. 4B shows another example method where, after dALE, selected isolates are characterized using a biosensor;

FIG. 5A is a schematic showing droplet merging for combining droplets of candidate with droplets containing biosensor cells;

FIG. 5B is a schematic showing co-encapsulation for combining candidate cells with biosensor cells;

FIG. 6 shows another schematic showing an example system for a functional essay utilizing a cellular biosensor with fluorescent protein reporter;

FIG. 7 shows a further schematic showing an example system for a functional essay utilizing a biosensor with an enzyme reporter;

FIG. 8A shows a method using dALE;

FIGS. 8B and 8C each shows an alternate method for identifying cells which produce desired products;

FIG. 9 is a bar graph of the relative production rates of high performing isolates at different scales. The rates of muconate production were normalized to the control cells (S4); and

FIG. 10 is a graph of muconate concentration vs. OD₆₀₀ showing selected isolates in solid circles.

### DETAILED DESCRIPTION

The method of the disclosure, droplet Adaptive Laboratory Evolution (dALE), was advantageously found to produce hardy cell cultures in about 2 weeks instead of the months-long timeframes needed when traditional adaptive evolution method is used. Encapsulating individual cells in microfluidic droplets is proposed to prevent cross-feeding or interference between different performers allowing only those cells that acquired positive traits due to spontaneous mutations thrive in the droplets. Cells that are not capable of dividing on their own remain dormant, while those with advantageous mutations can divide and form hundreds of daughter cells in a given droplet. When the droplets are broken and the cells are released, top performers are enriched in the mixture. In consecutive re-encapsulation, growth, and droplet breaking, the cultures are exponentially enriched with the fastest growers in a matter of weeks instead of months.

The disclosure is directed to efficient, low volume methods and systems for evolving cells which can flourish and replicate quickly in various environments. These techniques include droplet adaptive laboratory evolution (dALE). Identification of desirable cells using these techniques can be accomplished in a matter of days or weeks. Methods and systems associated with such techniques are provided, as well. Additionally, systems and methods for identifying hardy cells that produce a desired product are provided which utilize characterization techniques to indicate which cells are suitable. In some aspects, those techniques comprise cell-based based biosensors with either a fluorescent protein (FP) or an enzyme-linked reporter.

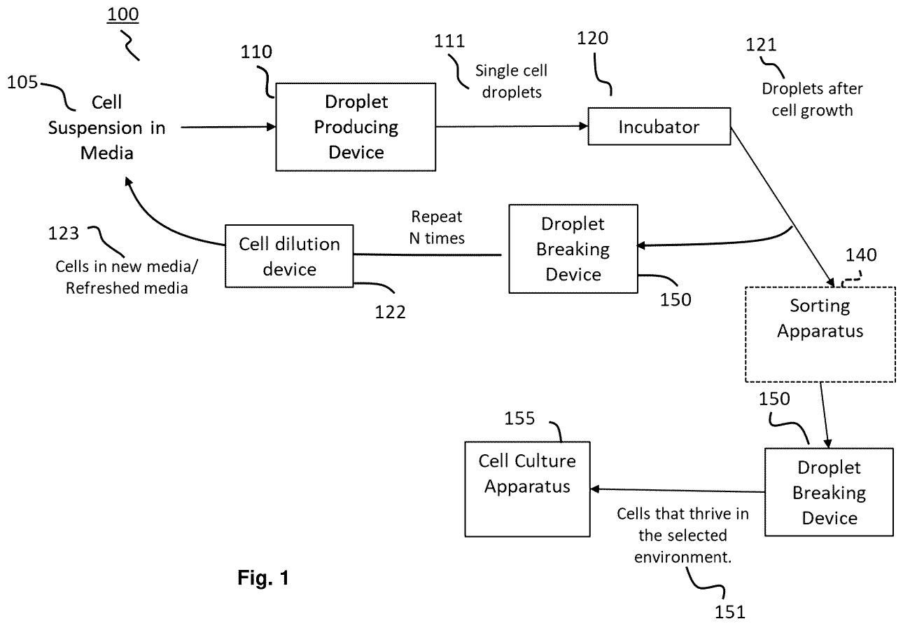

Turning to the Figures, FIG. 1 shows a system 100 for evolving cells and identifying those which exhibit hardiness and the ability to multiply quickly in selected mediums. In various aspects, the system 100 includes a cell suspension 105 (i.e., cells suspended in cell culture medium), a droplet producing device 110, one or more incubators 120, one or more droplet breaking devices 150, a cell dilution device 122, a cell culture apparatus 155, and optionally a droplet sorting apparatus 140.

Within the system, the types of products or information passed between components of the system 100 are shown as well. Types of products and information passed include any combination of single cell droplets 111, droplets after cell growth 121, cells in refreshed cell culture medium 123, and finally cells that thrive in the selected environment 151.

Droplet adaptive laboratory evolution (dALE) is useful in the methods described herein to evolve cells and produce isolates which thrive and reproduce quickly in a given environment while using the principle of survival of the fittest. Traditional ALE (adaptive laboratory evolution) has been used for evolving cells, but since it uses a mixture of candidate cell lines in medium-filled beakers or wells, the enrichment of top performers takes months because of interference between high and low performers. In contrast, dALE isolates cells in such a way that removes any effects that one cell line would have on another as they grow together. During multiple iterations of dALE, similar to iterations of ALE, spontaneous mutations occur in the cells. The dALE process favors cells which multiply quickly, as well as those best suited to thrive in a given environment (i.e. combination of cell culture medium and incubating environment). Further, the decreased medium volume when using dALE versus ALE is a resources saving.

Returning to the figures, the cell suspension 105 represented in FIG. 1 includes cells that are believed to be suited to survival in a chosen, or selected, environment. The initial suspension of cells includes any one of a genetically heterogenous or inhomogeneous cell population, a genetically homogenous cell population, natural cells, engineered cells, and a heterogeneous cell population generated by library approaches (e.g., transposon library, CRISPR-Cas9 library, cells expressing a library of enzyme or pathway variants in the engineered host). The selected environment includes the environment within the droplet provided by one or more cell culture media and an incubating environment characterized by variables external to the droplets. Environmental variables external to the droplets that form the incubating environment include any one or more of: percent oxygen (O₂), relative humidity, temperature, and agitation. The environment within the droplet is given by the cell culture medium in which the cells are suspended. The selected cell culture medium, in some aspects, includes any one of culture medium, minimal medium, selective medium, differential medium, transport medium, and indicator medium. In various aspects, the cell culture medium is of a specific pH, osmolarity, and/or ionic strength (i.e., salt content) which is challenging for certain cells. Alternatively, or additionally, the cell culture medium provides a specific type of nutrient, or feed-stock, e.g., starch, glucose. Other characteristics that may vary in the cell culture medium include an amount of, or presence of a toxin, cell debris, a biological product, waste, an inhibitor, and any combination thereof. In some aspects, the first cell culture medium and second cell culture medium are the same. In some aspects, the first cell culture medium and second cell culture medium are different. In some aspects, the cells present in the cell suspension 105 include preselected cells that are known to produce a desired product in the selected cell culture medium. A desired product can be, for example a chemical excreted into the media by the cell, including amino acids, plastic precursors, enzymes, or products that accumulate in the cells, for example oily droplets or combinations thereof. This cell suspension 105 of preselected cells in the selected medium is provided to the droplet producing device 110. In some aspects, the cells are selected to produce a desired product, and dALE process allows for selection of cells that are hardy and able to rapidly multiply in a cell culture medium that includes the desired product and impurities associated with the desired product. Exposure to the first and subsequent cell culture media results in a small subset of cells adapted to the various media and growth conditions (i.e. various selected environments), in some aspects due to accumulation of spontaneous mutations with positive effects.

The system and method include a droplet producing device 110. In some aspects, the droplet producing device 110 includes an active device, a passive device, traditional methods, ultrasonic droplet formation, atomization, a microfluidic chip-based method, off-chip methods, a process the proceeds from off-chip to on-chip then to off-chip, and a two-stream flow-focusing microfluidic device that accepts cells in the cell suspension 105. In some examples, the droplet producing device 110 is a two-stream flow-focusing microfluidic device that accepts cells in the cell suspension 105.

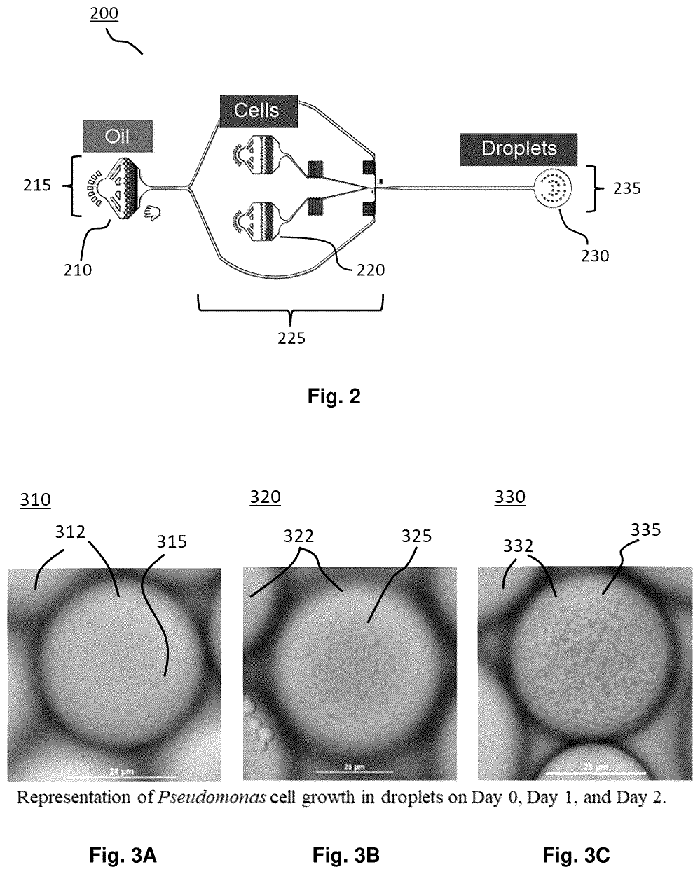

The droplet producing device can have a single-stream or two-stream flow-focusing option when mixing the cells with additional cell culture medium is desired or simply both channels are used for the introduction of cell suspension. The layout of a two-stream flow-focusing cell encapsulation PDMS chip 200 is shown in FIG. 2. In some aspects, the PDMS cell encapsulation chip 200 includes, or is fluidly connected to, a reservoir of oil 210 and a feature 215 on the chip 200 for directing oil flow on the chip 200. Further, the chip 200 shown in FIG. 2 includes a cell suspension reservoir 220 which feeds into a portion 225 of the chip 200 which draws up cells from the reservoir 220 and dilutes cells surrounded by the selected medium in oil from the oil reservoir 210. The configuration of the feature 225 of the chip 200 allows for the creation of droplets with a distribution of the number of encapsulated cells, with the majority of the distribution of encapsulated cells leads to droplets containing no more than one cell each. These droplets are captured at another location 235 on the chip 200 and collected in a collection reservoir or repository 230 before being placed in an incubator (120 in FIG. 1). Other configurations are possible for the cell encapsulation chip 200 as well as for the droplet producing device 110.

In such a droplet producing device 110 as shown in FIG. 2, droplets are formed from the cell suspension 105 by dilution of the cell solution with a fluorinated oil. The droplets are stabilized using a surfactant in the fluorinated oil, such as perfluoropolyether (PFPE). In such aspects, the droplets are created using a surfactant in the fluorinated oil.

Droplets produced by the droplet producing device 110 range in size from about 5 μm (microns) to about 2000 μm, about 10 μm to about 1000 μm, about 15 μm to about 500 μm, about 20 μm to about 100 μm, 30 μm to about 80 μm, such as from about 35 μm to about 75 μm, including from about 40 μm to about 70 μm, such as from about 45 μm to about 65 μm, and including from about 50 μm to about 60 μm. In some implementations, the droplets range in size from about 10 μm to about 150 μm in diameter, such as from about 15 μm to about 125 μm, including from about 20 μm to about 100 μm. Each droplet produced by the droplet producing device 110 has a volume less than 1 microliter, such as less than 1 nanoliter, including less than 900 picoliters, such as less than 900 picoliters, such as less than 800 picoliters, less than 700 picoliters, less than 650 picoliters, less than 600 picoliters, less than 550 picoliters, less than 500 picoliters, less than 450 picoliters, less than 400 picoliters, less than 350 picoliters, less than 300 picoliters, less than 250 picoliters, less than 200 picoliters, less than 150 picoliters, less than 100 picoliters, less than 90 picoliters, less than 80 picoliters, less than 70 picoliters, less than 60 picoliters, including less than 50 picoliters. In some implementations, each droplet has a volume of less than 45 picoliters. In various aspects, each droplet produced by the droplet producing device 110 has a volume of about 1 picoliters to about 1000 nanoliters, such as about 1 picoliter to about 900 nanoliters, about 2 picoliters to about 800 picoliters, about 3 picoliters to about 700 picoliters, about 4 picoliters to about 600 picoliters, about 5 picoliters to about 500 picoliters, such as about 25 microliters to about 35 microliters.

The droplet producing device 110 has a rate of encapsulation at least at 1000 droplets per second, capable of the production ranging from 1.0 million droplets to about 3 million droplets, such as from about 1.25 million to about 2.75 million droplets, including from about 1.5 million to about 2.5 million droplets from a predetermined amount of cell suspension 105 in less than an hour. In some aspects, the droplet producing device 110 has a rate of encapsulation of about 2.3 million droplets from about 200 μL (microliters) of cell suspension 105 in less than an hour.

Referring back to FIG. 1, the droplet producing device 110 passes droplets with either no cells (i.e., only the selected medium, cell-free medium) or a single cell 111 to an incubator 120 or incubation station. The incubator 120 allows the cells to grow within the droplets over time in the incubating environment. Incubation involves maintaining the collection of droplets in oil at the optimal temperature for the given organism (normally between 25 deg. C. and 38 deg. C.). Droplets are stable between 10 deg. C. to about 90 deg. C., which can be advantageous when dALE is used to adapt cells such as psychrophiles, mesophiles and thermophiles. dALE can be performed in either aerobic or anerobic conditions. In some aspects, the droplets in oil are gently agitated so as to provide constant aeration, or gas exchange, of the droplets. The extra fluorinated oil amended with the surfactant minimizes the oil-droplet emulsion from drying out. In some aspects, the surfactant can be present in an amount of about 2 wt. % to about 5 wt. %. In some aspects the incubating environment is the same in each dALE cycle. Alternatively, in some implementations, the incubating environment is different in one or more cycles.

Those cells which are better suited to replicating in the selected medium, i.e., those which adapt to the selected medium via genetic or epigenetic modifications produce more progeny cells over time within the droplet. FIGS. 3A-3C show the increase in the number of cells in a representative droplet over time. FIG. 3A shows an initial condition 310, in which a droplet 312 with a single cell 315 is shown surrounded by other droplets 312. In some aspects, the initial condition 310 is on the day that the droplet was formed, as well as when the medium within the droplet is fresh, and free from the products created by the cell. FIG. 3B shows a later condition 320. In the case shown in FIG. 3A-3C, the later condition 320 shown is after a day (i.e., 24 hours) has passed. The aging droplet 322 has an increased number of cells 325. FIG. 3C shows a droplet condition 330 at day two (i.e., 48 hours since starting the study). In FIG. 3C, the further aged droplets 332 are shown. One of the droplets 332 has a greater number of cells 335 within the aged cell 332 in the center. For example, FIG. 3A-3C show the progressive increase in Pseudomonas cells over time.

In the dALE process, after incubating the cells, the droplets are broken at a droplet breaking apparatus or station 150, and the contents of the droplets are accepted by the cell dilution apparatus 122 to admix a subset of the released cells with a first or a second cell culture medium forming a suspension of cells and provide this suspension of cells to the droplet producing device 110. The first cell culture medium and the second cell culture medium are the same, in some implementations. Alternatively, in some implementations, the first cell culture medium is different from subsequent cell culture media used to form the cell suspension. The initial environment and the subsequent environments may be the same or different. In some aspects, the initial environment and the subsequent environments differ in one or more constituents or characteristics. The cell dilution device 122 yields the cells from the droplet breaking device 150 suspended in new medium 123 (i.e., fresh or refreshed medium), ready for a re-encapsulation step or process. The cell dilution device 122 can select a subset of the cells, for example, those cells that grew most efficiently, for resuspension in the cell culture medium, thereby diluting out those cells that do not grow as efficiently. This cell suspension 105 is again presented to the droplet producing device 110, incubator 120 (as droplets), and the droplet breaking apparatus/station 150. This cycle, i.e., the incubating, breaking, resuspending and re-encapsulating steps is repeated multiples times, such that enrichment of the cells in suspension is observed. The number of repetitions of this cycle includes at least 2 times, at least 3 times, at least 4 times, at least 5 times, at least 6 times, at least 7 times, at least 8 times, at least 9 times, at least or about 10 times, at least or about 20 times, at least or about 30 times, at least or about 40 times, at least or about 50 times, at least or about 60 times, at least or about 70 times, at least or about 80 times, at least or about 90 times, and at least or about 100 times or more. In some aspects, the method for generating and/or selecting cells adapted to replicate in a selected environment, i.e., the dALE process is repeated about 4 to 10 times, such as about 5 to 9 times, including about 6 to 8 times in some implementations. In some aspects, the incubating, breaking, resuspending and re-encapsulating steps are repeated at least about 10 times over a period of about one to two months. The number of repetitions may be a predetermined number, or it may be based on a sampling of cells after droplet breaking 123. In some aspects, the cycling time depends on cell growth rate. For example, cells can reach a maximum cell density within 2-3 days, thus needing cycling every 2-3 days. Longer times are also contemplated herein, for example, for slower growing cells. In some instances of slower growing cells, the cycle time can be once a week or even once every other week. A suitable time can be selected based on cell growth rates for the given cell type being grown. Without intending to be bound by theory, it is believed that dALE can yield new phenotypes in timeframes of a few weeks, making it a robust method for adapting engineered cells to harsh environments.

The droplets are separated from the oil to collect the cells that are adapted to replicate in the selected environment following the iterations or repetitions of the growth and suspension in new media. In some implementations, the separation of the droplets and surrounding oil is accomplished using a membrane such as a fine mesh stainless steel membrane. Alternatively, in some implementations, an emulsion breaking solution is added to the droplets, resulting in an aqueous phase and an oil phase. The cell suspension may then be recovered from the aqueous phase.

The droplet breaking apparatus or station 150 includes one or more of any suitable device, apparatus, or assembly which removes the oil, or other liquid used to create the droplets, from the environs of the droplets. In some aspects, the droplet breaking device or apparatus includes a liquid capable of extracting and repartitioning a surfactant used in droplet formation. The product of the droplet breaking apparatus 150 includes candidate cells and/or representative of candidate cells. These cells are prepared and mixed with new medium.

Following a number of repetitions of the droplet formation and incubation process (dALE), the cells, which are no longer in droplets, are plated to obtain individual colonies, isolates are selected and cultured in growth medium. The growth medium is a suitable medium including growth medium in one or more well plates. This occurs after cells that thrive in the selected environment used in droplet formation and incubation are passed to a cell culturing apparatus 155.

The cells which are cultured represent cells which were able to grow in the selected environment. In the dALE process, as individual cells are encapsulated with each iteration of the droplet formation and incubation process, each cell that is able to grow in significant numbers in the selected environment may be present, with those better suited to growth in the selected environment being present in greater numbers. The dALE process favors the proliferation of cells which are able to quickly reproduce and thrive in the selected environment, as the period between cell encapsulation and droplet breaking is relatively short, such as less than 10 days, less than 9 days, less than 8 days, including less than 6 days. In some implementations, the period of time between cell encapsulation and droplet breaking in the dALE process is between 12 hours and 5 days, including between 1 day and 4 days, such as between 2 and 3 days.

In an example of how dALE (droplet adaptive laboratory evolution) will come to select the cells that are best suited to thriving in a selected environment, a cell of strain X may grow well resulting in 200 cells in a droplet after a first iteration of the droplet and incubation process. These 200 cells are then encapsulated into their own droplets, one cell per droplet, at the start of the second iteration. At the end of the second iteration, cell X will be represented by at least 40000 cells in the overall suspension. Then, at the end of the third iteration, assuming that each of the 40000 cells, each in its own droplet, divide resulting in 200 cells per droplet, there will be 8 million cells of strain X resulting from the initial droplet. If strain Y grows slower resulting in, for example, 50 cells per droplet for each iteration, beginning with one cell of strain Y in a single droplet, after three iterations, strain Y will be represented by 125000 cells in the overall suspension. Then, at the end of three iterations of dALE cell cultivation, the number of cells of strain X will outnumber those cells of strain Y by 64:1. Most importantly, a cell of strain Z may not be able to grow at all in the given condition, resulting in 3 cells at the end of round 3 and therefore being practically eliminated from the population.

As shown in FIG. 1, following a number of repetitions (N repetitions, as described herein above) of the droplet formation and incubation process (i.e., dALE), the droplets are optionally provided to a sorting apparatus 140. The sorting apparatus 140 sorts droplets based upon a presence or an absence of an analyte or desired product, such as is indicated or detected with a biosensor.

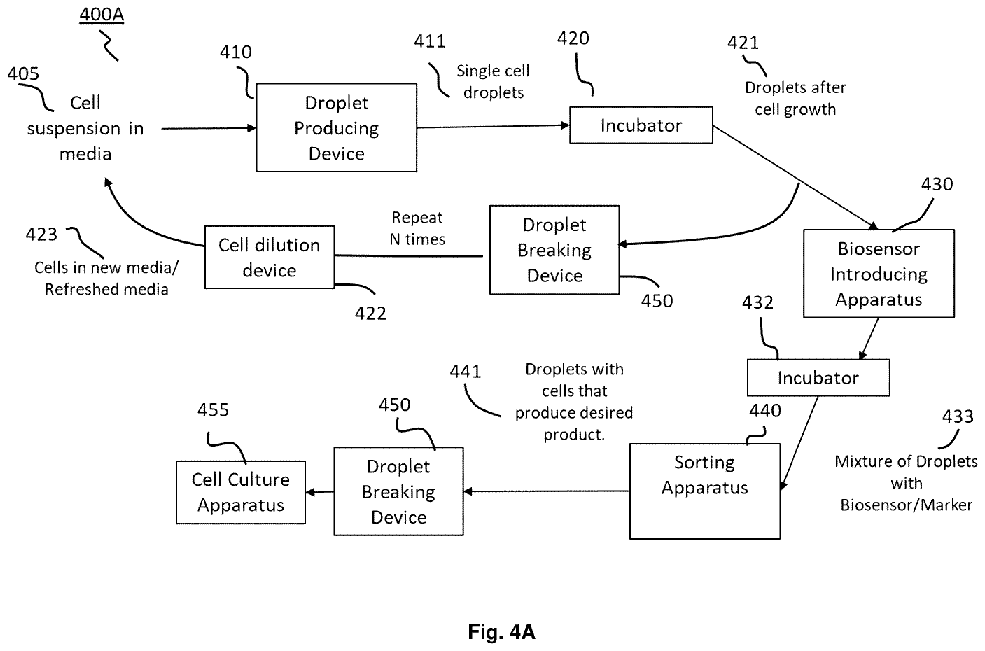

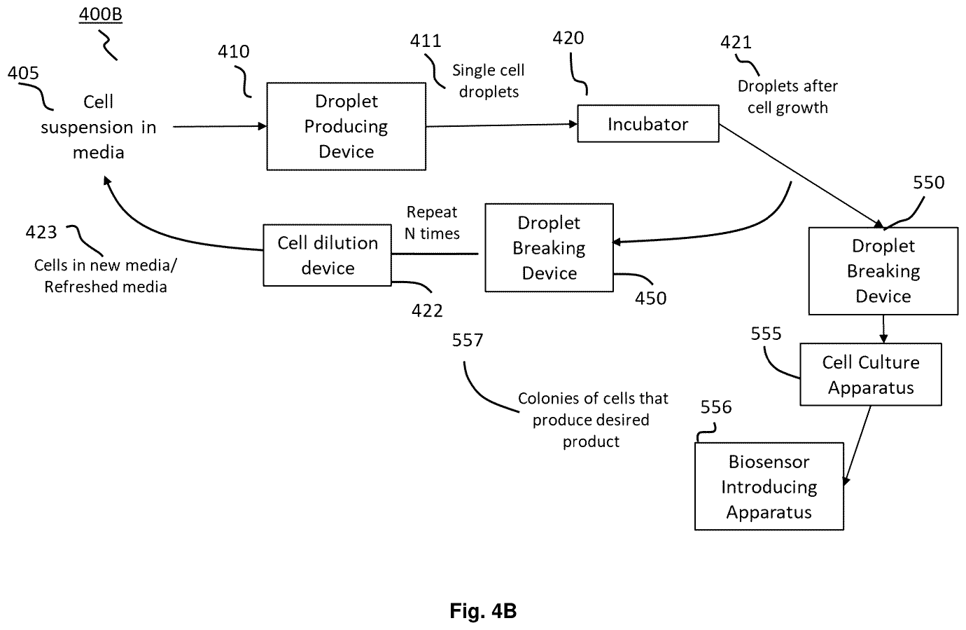

FIG. 4A and FIG. 4B illustrate systems which utilize the dALE process in conjunction with a biosensor to identify cell lines which produce a desired product or analyte. In these processes 400A and 400B, a suspension 405 of starter or candidate cells in a selected environment is provided. As indicated above, the first cell suspension includes cells pertaining to strains known to produce the desired product in some implementations. From the suspension 405, no more than one cell is encapsulated 410 per droplet 411 produced by a droplet producing device 410. That is to say, the droplet producing 410 device may create droplets that contain only medium and that contain no cells or there may be a distribution in the number of cells in each droplet with the mean number of cells in each droplet being 1 cell. The droplets 411 are then sent to an incubator 420 and incubated for a preselected number of days (e.g., 2 days), yielding droplets with varying number of cells 421. Cells which are well suited to the environment or that mutated to thrive in the environment will have multiple cells per droplet. The droplet breaking device 450 or apparatus frees the cells from their droplets. The freed cells are diluted by device 422 with medium 423 and this suspension 405 is provided as the start of a subsequent iteration of the dALE process 400A, or 400B. As indicated above, the number of times (i.e., iterations) the process 400A, or 400B is repeated is either a preset number or depends on the growth of the cells within the droplets. The number of iterations of dALE is constrained by a time period. In some implementations, the time between iterations is 4 days or less, and the total number of iterations is 10, such that the total amount of time for such an implementation is 40 days. The time between iterations, in some aspects, is 6 days or less, including 5 days or less, 4 days or less, 3 days or less, and 2 days or less, and the amount of total time for implementation is 14 days or less, 10 days or less, including 8 days or less.

In some cases, as shown in FIG. 4A, once the dALE process has been repeated or iterated upon a given number of times, the cells which were freed from their droplets are formed into droplets which will include a biosensor cell 441 that will help discern which cells produce the desired product as well as thrive in the selected medium.

Alternatively, in some cases, as shown in FIG. 4B, after N repetitions of the dALE process, the droplets 421 are provided to the droplet breaking device 550. The cell culture apparatus 555 receives the surviving cells which are collected from the isolated colonies on an agar plate using cells obtained from broken droplets after the final round of dALE. The surviving cells are grown and then the biosensor introducing apparatus 556 provides one or more biosensors to colonies of the surviving cells. The biosensor(s) identify colonies 557 of cells grown from the surviving cells that produce the desired product.

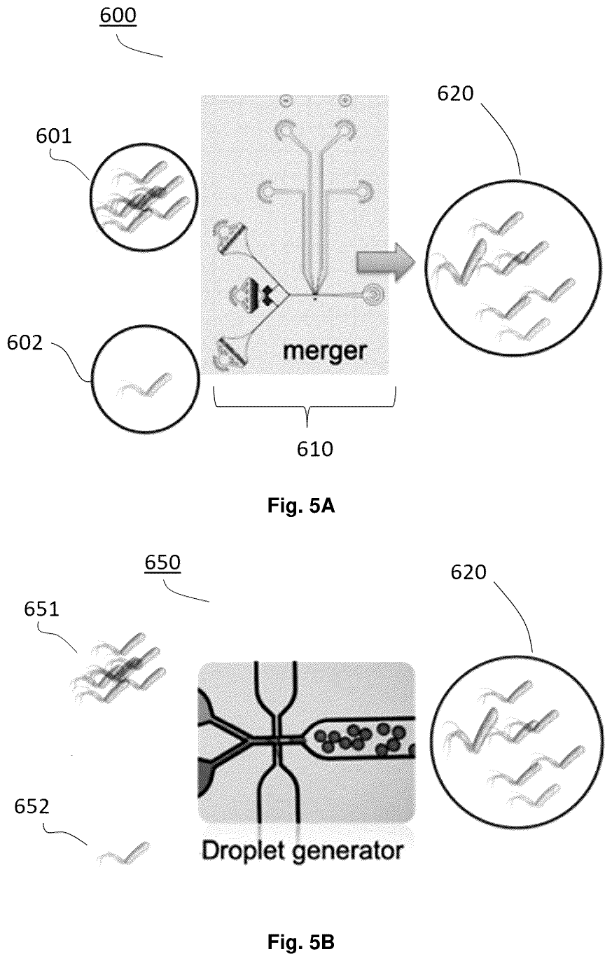

FIGS. 5A and 5B show the creation of droplets which contain both candidate cells and at least one biosensor cell using two different approaches. FIG. 5A is a schematic of droplet merging 600. FIG. 5B is a schematic showing co-encapsulation 650 of the candidate cell(s) and at least one biosensor cell.

In FIG. 5A, in which droplet merging 600 is used, the candidate cells 601 are identified as P. putida cells. Single candidate P. putida cells are encapsulated in a first droplet and incubated. The cells divide and produce the desired product in 2-3 days (hundreds of cells per droplet and product titer reached maximum). The biosensor cell 602, shown as an E. coli cell, is encapsulated in a second droplet 620. A merging apparatus 610 is used to create a single droplet 620. The nature of droplet merging yields droplets which are of varying size and volume, depending on the ability of the apparatus to coordinate the flow of droplets containing candidate cells and those containing biosensors. In some aspects, the diameter (i.e., size) of the droplets ranges from about 0.9 microns (μm) to about 2000 microns, about 1.0 micron to about 1000 microns, about 1.5 microns to about 750 microns, about 2.0 microns to about 500 microns, about 3.0 microns to about 400 microns, about 4.0 microns to about 300 microns, about 5.0 microns to about 200 microns, about 10 microns to about 100 microns, such as from about 20 microns to about 90 microns, such as from about 30 microns to about 80 microns, including from about 40 microns to about 70 microns and from about 50 microns to about 60 microns. In some implementations, the droplet diameter ranges from about 5 microns to about 2000 microns.

FIG. 5B is a schematic of co-encapsulation 650. The starting materials for the co-encapsulation process include two types of cell suspensions. The first is a suspension of candidate cells 651 which have been through the dALE process multiple times. The second is a suspension of biosensor cells 652. Droplets 620 containing both the candidate cells and biosensor cells are formed by a droplet producing device. The droplet producing device, in some aspects, is similar to that used during the dALE process. Alternatively, another apparatus or protocol may be used to form droplets containing cells from both of these cell suspensions.

In addition to using droplet merging and co-encapsulation to obtain droplets containing both candidate cells and a biosensor, a biosensor may be injected into an existing droplet that contains candidate cells, such as by using pico-injection. An apparatus for pico-injection includes a chip with any of a flow channel through which the existing droplets pass, and/or a cross-channel or opening in the flow channel that allows for a minute amount of fluid containing the biosensor to be introduced into the existing droplet.

In some aspects, the biosensor is a sensor cell which produces a fluorescent compound (e.g., green fluorescent protein) in the presence of the desired biological product, such that the luminesce is proportional to the amount of the desired product is present. The biosensor may include a sensor cell with an enzyme linked variant. The use of a sensor cell with an enzyme linked variant may create a more uniform distribution of fluorescence in a volume of liquid.

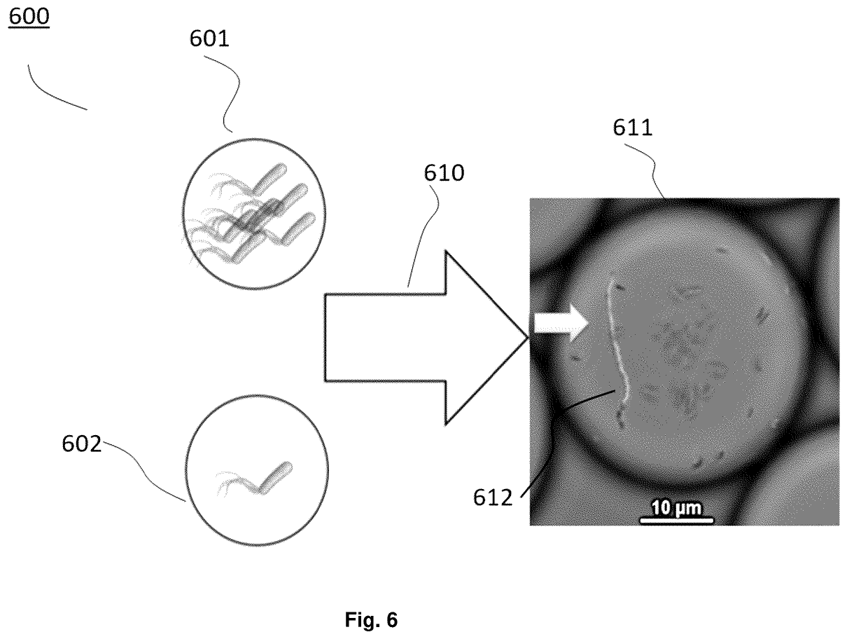

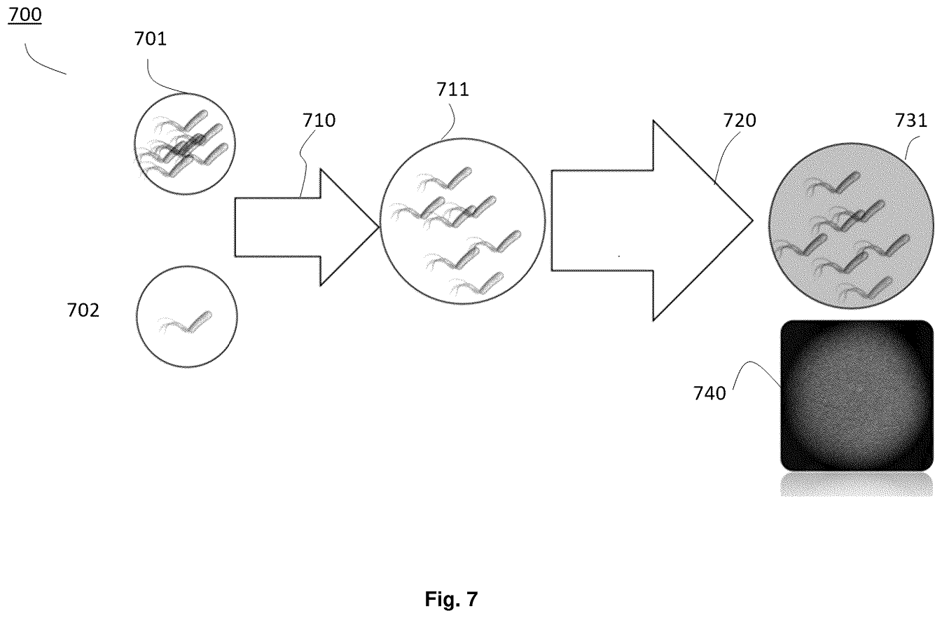

FIG. 6 and FIG. 7 illustrate using a cell-based biosensor with an FP reporter 600 and a cell-based biosensor with an enzyme-linked variant 700, respectively. In each of the figures, the initial condition of the candidate cells is that they are encapsulated in a droplet 601, 701. In these figures, the candidate cells are P. putida cells which have been through the dALE process multiple iterations, such that there are one or more P. putida cells per droplet. The initial condition of the biosensor cells (i.e., E. coli sensor cells) is that they are encapsulated, one sensor cell per droplet 602, 702. These figures show the combination of these droplets via droplet merging or pico-injections 610, 710. In FIG. 6, the merged droplet 611 is then incubated and the biosensor is allowed to interact with the products of the candidate cells, yielding a droplet with a concentrated area of green fluorescent protein 612 which is visible in response to scanning with light of a particular wavelength. In contrast, FIG. 7 shows that the merged cell 711 is incubated, then the biosensor cells are lysed 720 so that the enzymatic support may enable more even distribution of fluorescence 731 when in the presence of the desired product. A droplet with such an even distribution of fluorescence 740 may be easier to detect for droplet sorting or direct comparison between droplets that initially contained both candidate cells and biosensor cells.

Once droplets have been through the dALE process numerous iterations, droplets with one or more candidate cells are passed to the biosensor introducing apparatus 430 in FIG. 4A. As described with respect to FIGS. 5A-7, there are multiple ways of introducing a biosensor into each droplet, as well as multiple types of biosensors. In the implementation shown in FIG. 4A, the biosensor introducing apparatus 430 creates a number of droplets with both candidate cells and biosensors. The biosensor cells are introduced either by picoinjection or via droplet merging. These droplets are then provided to an incubator 432 so that the biosensor can react with the products of the candidate cells. After incubation, the droplets 433 are provided to a sorting apparatus 440. In some aspects, the sorting apparatus 440 utilizes fluorescence-activated droplet sorting (FADS). Fluorescence-activated droplet sorting includes the use of one or more electrodes. The sorting apparatus 440 yields droplets that enclose cells that produce the desired bioproducts 441. The sorting apparatus 440 may have a threshold above which a droplet is selected for further processing by the droplet breaking device 450. For example, in an approach, where fluorescence-activated droplet sorting is employed, droplets which do not display a threshold amount of fluorescence after being probed with light of an appropriate wavelength may be rejected.

The selected droplets are then passed to droplet breaking device 450. This droplet breaking device may be the droplet breaking device 450 used during the dALE iterations, a similar device, or an entirely different device. The droplet breaking device 450 then passes the cells which are no longer in droplets to a cell culturing apparatus 455.

The cells provided to the cell culturing apparatus 455 are those which were identified as producing the desired product, and these cells are cultured in growth medium. The growth medium may be any suitable medium including agar in petri dishes and/or liquid growth medium in one or more well plates. The cells which are cultured represent cells that were able to rapidly replicate in the selected environment. As individual cells are encapsulated with each iteration of the droplet formation and incubation process, each cell that is able to proliferate in significant numbers in the selected environment are present, with those better suited to replication in the selected environment being present in greater numbers, as described above.

FIG. 4B shows a system similar to that shown in FIG. 4A, in that both utilize the dALE process to obtain cells that are well suited to rapid replication in the selected environment. In FIG. 4B, following the dALE process, the droplets are provided to a droplet breaking device 550, and a cell culture apparatus 555 receives those cells. The cell culture apparatus 555 produces colonies from the cells received from the droplet breaking device. To these colonies, a solution with at least one biosensor is introduced via the biosensor introducing apparatus 556. Colonies of cells that produced the desired product 557 result from the processes of the biosensor introducing apparatus 556. After cell cultures have been allowed to grow for some days or until a threshold amount of growth is observed, an aliquot of the medium and products surrounding each cell isolate is extracted and placed into an array, such as in a well of a well plate. The samples or aliquots are diluted, in some aspects, after extraction. The arrays may be screened, evaluated, or probed to determine the amount of the desired product produced by each cell isolate. The cells which are determined to replicate rapidly and produce a desired biological product above a threshold level are collected, in some aspects for further characterization or experimentation.

In some approaches, the arrays may be created at set time intervals. This periodic sampling of the products of each of the cell isolates may enable their proliferation and ability to produce a desired bioproduct to be tracked over time. A curve plotting the relative amount of product (e.g., intensity fluorescence, spectroscopic intensity data) versus time includes an inflection point which may be indicative of a plateau in the ability of a strain to produce the desired biological substance. This information may be useful when producing large volumes of the desired biological substance or product. The combination of dALE and the periodic analysis of products from the cell isolates enables the most suitable cells to be identified within weeks to months, as opposed to over the course of multiple months to a year when using approaches using large volumes of medium and conventional incubation methods.

The instrument or system used to determine the amount of the desired biological substance present in each aliquot, or a relative amount, includes any of a chromatography technique or instrument, a mass spec technique or instrument, a fluorescence scanner, the use of biosensors, a blotting technique or instrument, or any combination thereof.

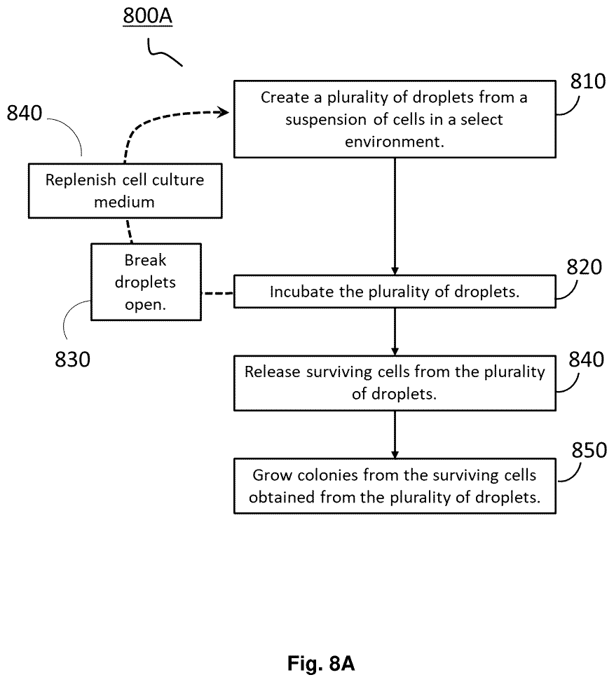

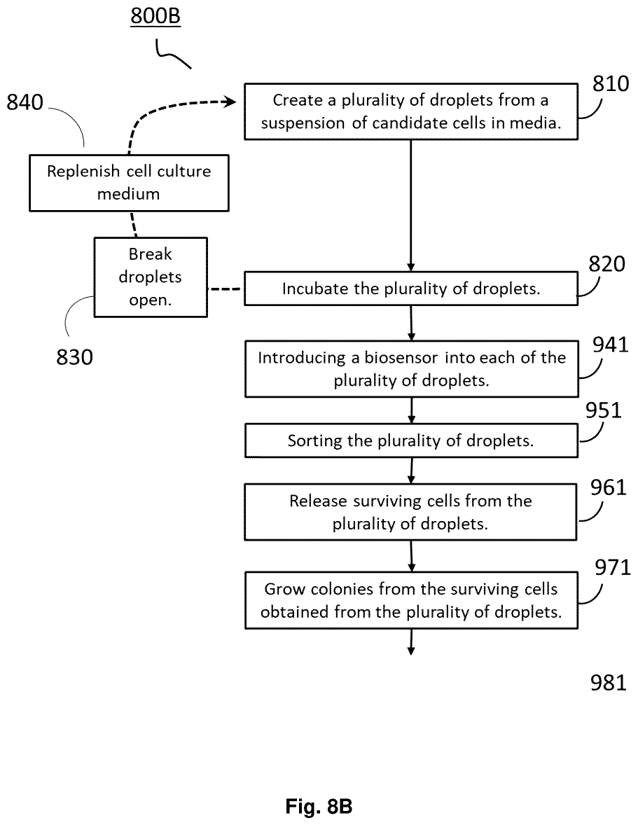

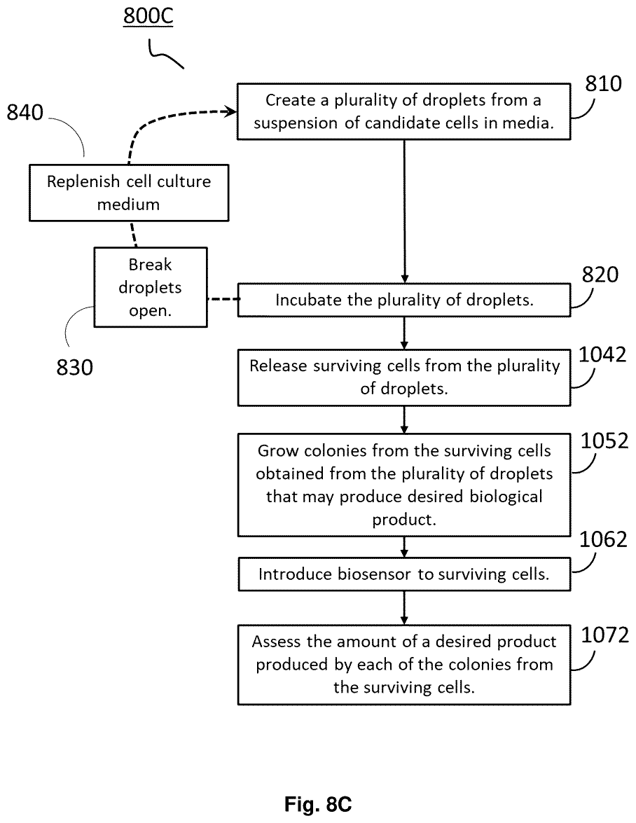

FIGS. 8A-8C each shows an example method 800A, 800B, 800C for use of the systems and processes described above.

FIG. 8A shows a method 800A for determining cells which are hardy and quickly multiply in selected medium(s). The method 800A starts with the creation or provision of a plurality of droplets from a suspension of cells in a selected environment, as in step 810. The plurality of droplets each have no more than one cell. Following droplet creation, the method includes incubation, as in 820. As described above, with respect to the dALE process, incubation of the droplets is for a predetermined amount of time. In some implementations, incubation is for a time period of about 3 days. In some aspects, however, the incubation time period is from about 1 day to about 10 days, from about 2 days to about 9 days, from about 2 days to about 8 days, from about 2 days to about 7 days, from about 2 days to about 6 days, from about 2 days to about 5 days, from about 2 days to about 4 days, or from about 3 days to about 4 days. During that time, droplets with cells which are well suited to the selected environment may reproduce within their droplet, while those not suited to the environment fail to replicate or even die. After the incubation period, the droplets are broken open as in 830, and the surviving cells are released. After this, the retrieved cells are diluted, that is fresh medium is added to the suspension, so that droplets may be formed again as in 810. This cycle of droplet formation, where each drop encapsulates no more than one candidate cell, dALE, is performed multiple times, or iterations, as indicated hereinabove, for example such as from 5-10 times, including from 6-8 times.

Following multiple iterations of the dALE process, the surviving cells are released from the plurality of droplets, as in step 840. Colonies are grown from the cells resulting from the dALE process as in 850. When determining cells which thrive in a selected environment and produce a desired biological product, in the method 800A, supernatants from the cell colonies are assayed to determine the presence of the desired product, and in some aspects these assays may include determining the relative amount of the desired product. Chemical characterization methods and apparatus are used to determine the presence of the desired product. Such methods include mass spectroscopy, chromatography, quadrupole time of flight (TOF) mass spectroscopy, Raman spectroscopy, UV-vis spectroscopy, and the like.

The method 800B shown in FIG. 8B begins in the same way as in method 800A. Following the desired iterations of droplet creation and incubation, the next step is to introduce a biosensor into each droplet as in 941. In some aspects, the introduction or insertion of the biosensor involves any one of droplet merging, pico-injection of biosensor cells, or co-encapsulation, as described above. In cases where the introduction of the biosensor includes co-encapsulation, the introduction step may involve creating separate suspensions of the candidate cells and the biosensor cells which are provided to a droplet forming apparatus. After the biosensors and candidate cells are encapsulated in the same droplet and incubated, the biosensor indicates the presence, and in some instances, the relative amount of the desired product.

Once the biosensor has interacted with the products of the candidate cells the droplets are sorted, as in 951. In some aspects, the sorting is simply based on the detection of the presence of the desired product. Alternatively, in some aspects, the sorting is based on a degree to which a desired product is detected. Cells from the positively selected droplets are then released as in 961 and then cultured, forming colonies, as in 971. A liquid handling apparatus samples supernatant from the cultures from the cell isolates and creates an array of aliquots that is representative of the cells obtained from the positively selected droplets. The supernatant is diluted with another liquid prior to further evaluation. The array of aliquots are then tested or sampled to determine the relative amount of the desired product generated by each cell or colony as in 981. In some aspects, the cells identified to both replicate rapidly and produce the desired product are collected for future use.

The method 800C shown in FIG. 8C begins as in methods 800A and 800B with the dALE process. Following the dALE process, the method includes the release of surviving cells from the plurality of droplets as in 1042. Colonies are grown from the surviving cells which may produce the desired product as in 1052. To the colonies, the biosensor is added as in 1062. The biosensor are cells or a cell derivative, such as an enzyme in some aspects. The presence, and possibly the amount, of a desired product produced by each of the colonies obtained from the survivor cells are assessed as in 1072. As with the methods 800A and 800B, the most successful cells with respect to both bioproduct production and proliferation rate are collected for future use, in some implementations.

The systems and processes described herein utilize dALE to determine cells which are robust and hardy in selected environments, as well as those which proliferate quickly in those environments. The systems and processes are used to identify which of these robust, hardy, and quickly multiplying cells also produce a desired product.

### EXAMPLES

The following examples are provided for illustration and are not intended to limit the scope of the invention.

In general, adaptive laboratory evolution (ALE) is commonly used to acclimate engineered strains to realistic biomanufacturing environments, generating evolved isolates with higher feedstock and/or product tolerance, or for increased resistance to impurities in sugar streams generated from lignocellulosic feedstocks, while improving performance as measured by either titers, rates, and/or yields.

The advantages of miniaturized, droplet-based microfluidics for adaptive laboratory evolution (dALE) were tested by encapsulating and growing individual cells in droplets of about 50 μm in diameter which resulted quickly enriched in top-performing cells. The dALE process was carried out as described, by periodic breaking of the emulsions and the re-encapsulation of single cells and resulted in isolates with faster growth on the given feedstock within 2 weeks. Small-scale evaluation of isolates via automation and HTP-MS methods capable of analyzing more than 500 samples per day was also demonstrated.

Testing was performed at small scale with inoculum derived from pL droplets (1 mL in deep 96-well plates). Evaluation was scaled thereafter to 1 L (flasks or bioreactors) to validate that growth and productivity phenotypes of the isolates were conserved and were ready to be used as production strains.

For the series of runs produced according to the conditions listed in Table 1, the dALE method was deployed using engineered P. putida strains, namely strains: S1, S2, S3, and S4. Table 1 includes the growth conditions along with the DBTL cycle improvement metrics.

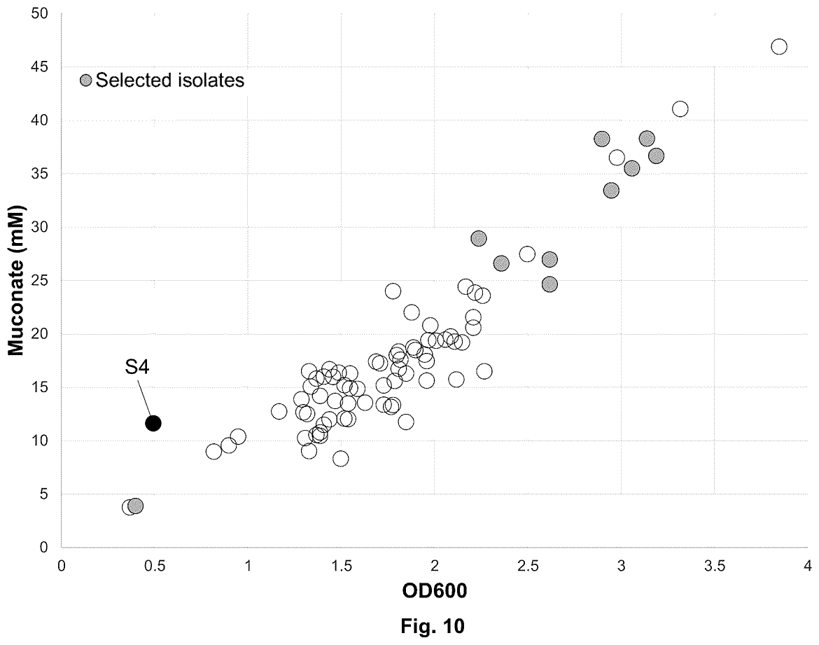

Isolates were tested for growth by cell density using light scattering at 600 nm (OD₆₀₀) and muconate titers. Top performers were selected from the top right quadrant of a muconate concentration vs. OD₆₀₀ plot (FIG. 10) and the corresponding top performing isolates evaluated in 1-mL scale in triplicate or quadruplicate. These experiments performed at a pL-derived screen employed a high throughput (HTP) analytical strategy enabling the rapid monitoring of growth and muconate production. The HTP analytics were performed by the Selected Ion Monitoring (SIM) mass spectrometry method. Growth of the initial cultures in the pL screen was not fully harmonized, potentially yielding cultures with lower initial densities and apparently poor performance. The combination of fast analytics and incomplete harmonization of cultures was observed to produce noisy data and lead to some isolates in the pL screen identified as false negatives (for example, S4). S4 was found to be a good performer when evaluated at the 1-mL setting.

TABLE 1

| | Run | | | | | | |
|---|---|---|---|---|---|---|---|
| | 1 | 2 | 3 | 4 | 5 | 6 | 7 |
| Strain | S1 | S1 | S2 | S3 | S4 | S4 | S4 |
| dALE | M9- | M9- | M9- | M9- | M9- | M9- | M9- |
| | glucose | DMR | DMR | DMR | DMR | DMR | DMR |
| | (30 | (30 mM | (30 mM | (30 mM | (30 mM | (90 mM | (270 mM |
| | mM) | glu) | glu) | glu) | glu) | glu) | glu) |
| Isolates | 92 | 92 | 180 | 180 | 88 | 88 | 74 |
| selected | | | | | | | |
| Top | 15 | 9 | 3 | 15 | 12 | 8 | 28 |
| performers | | | | | | | |
| Shake flasks | 5 | 5 | 5 | | | | |
| Bioreactor | 0 | 1 | | 2 | | | |
| Genotyping | 0 | 12 | 5 | 10 | | | |

| | | | | | |
|---|---|---|---|---|---|
| Initial mini- | 14 weeks | 9 weeks | 11 weeks | 5 weeks | 14 weeks |
| DBTL | (0.9/d) | (1.5/d) | (4.7/d) | (5.6/d) | (1.1/d) |

One isolate (T11) demonstrated improved production rate over the engineered parent stain (S3), and the performance benefit was maintained when scaled from pL droplets to 0.5 L bioreactor scale, where a 3-fold improvement was maintained (0.22 g/L/h vs. 0.07 g/L/h) using DMR-EH (15 g/L total sugars). DMR-EH represents the sugar hydrolysate obtained by deacetylation and mechanical refining followed by enzymatic hydrolysis of the mechanically and acid/base pretreatment of the lignocellulosic feedstock.

Additional testing was performed to adapt the advanced S4 strain (a ‘clean’ engineered mimic of S3) to higher DMR-EH concentrations. Specifically, cells were evolved in 90 mM glucose-equivalent DMR-EH (Run 6 in Table 1). At this DMR-EH concentration, the S4 parent strain showed limited growth whereas the evolved isolates reached high cell densities and increased muconate titers and rates, while maintaining yields.

Ten isolates were used to compare the performance of the isolates with the parent (S4). In particular, the comparison was done in deep 96-well blocks with 1 mL culture using inoculum derived from pL droplets and at 1 L (2.8 L Fernbach flask) scale with inoculum from a freezer stock in parallel. To enable successful comparison with the parent (which grows poorly in high sugar concentrations), 30 mM glucose equivalent mock hydrolysate was used. The isolates displayed shorter lag times in the growth phase compared to the parent strain (S4) and S3 at both scales. Muconate production displayed a similar trend reaching 9-11 mM titers more than 15 hours earlier than the parent, in addition, glucose and total C5 sugars were more rapidly utilized in the evolved isolates compared to S4 and S3.

The isolates at 1-mL scale were evaluated in quadruplicates. A tight clustering of isolates was observed, partly due to the emphasis on the top performers in Campaign 6 for downstream studies. The narrow range of performance differences between the isolates across scales made it difficult to establish a ranked order. Instead, a percent improvement of rates compared to the S4 parent were calculated. These normalized values made it possible to assess the effect of scaling to 1 L.

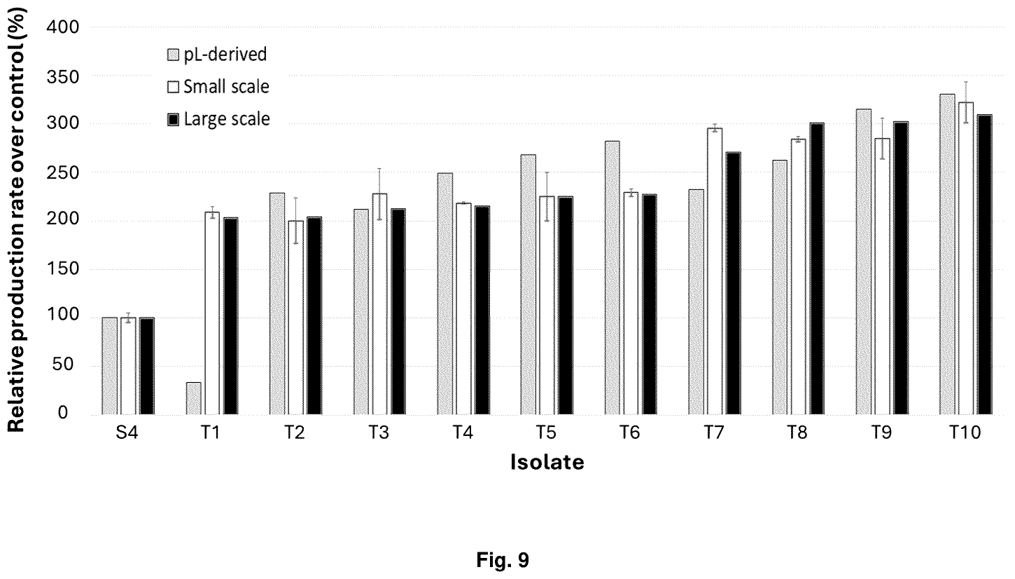

Advantageously, it was found that the isolates from the scale-up trials showed over 2-fold and up to 3-fold improvements in production rates combined with a retention of at least 80% of the isolates' performance increase (Table 2). The production rates of large-scale (1-L) cultures were similar to those of the small-scale (1-mL) experiments. The performance of the isolates relative to the parent strain were also very similar at both scales (FIG. 9). These results suggest that the evolved isolates can retain improved performance as they approach relevant biomanufacturing scales, despite the miniaturization of ALE at the initial development stages and throughout the scale-up process.

TABLE 2

| | pL screen | | mL confirmation | | L validation | |
|---|---|---|---|---|---|---|
| | | Percent of | | Percent of | | Percent of |
| | Rate | control | Rate | control | Rate | control |
| Isolate | (mg/L/h) | (S4) | (mg/L/h) | (S4) | (mg/L/h) | (S4) |
| S3 | | | 9.4 | 57.6 | 11.9 | 74.3 |
| S4 | 36.8 | 100.0 | 16.3 | 100.0 | 16.1 | 100.0 |
| T1 | 12.4 | 33.7 | 34.1 | 208.8 | 32.6 | 203.1 |
| T2 | 84.2 | 228.8 | 32.6 | 199.9 | 32.7 | 203.9 |
| T3 | 78.0 | 212.0 | 37.1 | 227.5 | 34.1 | 212.4 |
| T4 | 91.5 | 248.6 | 35.6 | 218.3 | 34.5 | 215.0 |
| T5 | 105.8 | 287.5 | 36.7 | 224.7 | 36.1 | 224.9 |
| T6 | 121.1 | 329.1 | 37.4 | 229.1 | 36.5 | 227.2 |
| T7 | 85.4 | 232.1 | 48.2 | 295.2 | 43.4 | 270.3 |
| T8 | 121.2 | 329.3 | 46.3 | 283.8 | 48.3 | 300.7 |
| T9 | 116.1 | 315.5 | 46.4 | 284.5 | 48.6 | 302.5 |
| T10 | 112.4 | 305.4 | 52.5 | 321.8 | 49.6 | 309.1 |

Full genome sequencing of isolates and parents using a hybrid Nanopore and Illumina sequencing approach revealed not only single nucleotide polymorphisms (SNPs) but also potential larger genome rearrangements as a consequence of dALE that correspond with the growth of cells with an increased capacity to survive in the selected environment at each iteration of the dALE process.

It was found that a combination of a short dALE (<3 weeks), a rapid pL-screening (<1 week), followed by careful characterization in small scale (<4 weeks) was surprisingly and advantageously useful in identifying top isolates that retain their improved performance at large scale in bioreactors.

Without intending to be bound by any theory, it is believed that isolates generated via dALE develop tolerances to feed-stock components and/or products that result in increased production rates without decreases in titers or yields. These improvements persist in large-scale cultures. The following additional considerations apply to the foregoing discussion. Throughout this specification, plural instances may implement functions, components, operations, or structures described as a single instance. Although individual functions and instructions of one or more methods are illustrated and described as separate operations, one or more of the individual operations may be performed concurrently, and nothing requires that the operations be performed in the order illustrated. Structures and functionality presented as separate components in exemplary configurations may be implemented as a combined structure or component. Similarly, structures and functionality presented as a single component may be implemented as separate components. These and other variations, modifications, additions, and improvements fall within the scope of the subject matter herein.

As used herein any reference to “some examples” or “one example” or “an example” means that a particular element, feature, structure, or characteristic described in connection with the example is included in at least one example. The appearances of the phrase “in one example” in various places in the specification are not necessarily all referring to the same example.

Some examples may be described using the expression “coupled” and “connected” along with their derivatives. For example, some examples may be described using the term “coupled” to indicate that two or more elements are in direct physical or electrical contact. The term “coupled,” however, may also mean that two or more elements are not in direct contact with each other, but yet still co-operate or interact with each other. The examples are not limited in this context.

As used herein, the terms “comprises,” “comprising,” “includes,” “including,” “has,” “having” or any other variation thereof, are intended to cover a non-exclusive inclusion. For example, a function, process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the examples herein. This is done merely for convenience and to give a general sense of the description. This description should be read to include one or at least one and the singular also includes the plural unless it is obvious that it is meant otherwise.

Upon reading this disclosure, those of skill in the art will appreciate still additional alternative structural and functional designs for methods and systems for producing a reusable structural member through the disclosed principles herein. Thus, while particular examples and applications have been illustrated and described, it is to be understood that the disclosed examples are not limited to the precise construction and components disclosed herein. Various modifications, changes, and variations, which will be apparent to those skilled in the art, may be made in the arrangement, operation and details of the method and apparatus disclosed herein without departing from the spirit and scope defined in the appended claims.

The following list of aspects reflects a variety of the examples explicitly contemplated by the present application. Those of ordinary skill in the art will readily appreciate that the aspects below are neither limiting of the examples disclosed herein, nor exhaustive of all the examples conceivable from the disclosure above but are instead meant to be exemplary in nature.
1. A method for generating and/or selecting cells adapted to replicate in a selected environment, the method comprising steps of:
encapsulating cells from a first suspension of cells in a first cell culture medium into a plurality of droplets, each droplet comprising one or more cells from the initial suspension of cells;
incubating the plurality of droplets in an incubating environment;
breaking the plurality of droplets to release cells;
resuspending a subset of the released cells in a second cell culture medium to form a second suspension of cells and
re-encapsulating cells from the second suspension of cells to form a plurality of droplets, each droplet comprising one or more cells from the second suspension of cells;
wherein:
the selected environment includes one or more of the first cell culture medium, the second cell culture medium, and the incubating environment; and
the incubating, breaking, resuspending and re-encapsulating steps are repeated to identify cells adapted to replicate in the selected environment.
2. The method of aspect 1, wherein the incubating environment comprises any one or more of: percent oxygen (O₂), relative humidity, temperature, and agitation.
3. The method of aspect 1 or 2, wherein the first culture medium and/or the second cell culture medium comprise:
a feed-stock;
a toxin;
an inhibitor;
a salt;
a selected ionic strength;
a selected osmolarity;
a biological product or byproduct;
a media component; and
cell debris/waste.
4. The method of any one of aspects 1-3, wherein the incubating, breaking, resuspending, and re-encapsulating steps are repeated at least 4 times.
5. The method of any one of aspects 1-4, wherein the incubating, breaking, resuspending, and re-encapsulating steps are repeated at least 2 times in a time period of seven days.
6. The method of any one of aspects 1-5, wherein incubating the plurality of droplets is done over a period of time equal to at least 12 hours.
7. The method of any one of aspects 1-6, further comprising collecting the identified cells that are adapted to replicate in the selected environment and optionally further comprising evaluating the cells adapted to replicate in the selected environment based on ability to produce a desired biological product.
8. The method of any one of aspects 1-7, further comprising:
introducing a biosensor to the plurality of droplets, wherein the biosensor emits a signal when the desired product produced by the cells is present; and
sorting the plurality of droplets based upon a presence or an absence of the biosensor signal in the droplet.
9. The method of aspect 8, wherein introducing the biosensor comprises any of:
injecting the biosensor into each droplet of the plurality of droplets using a pico-injection method;
co-encapsulation of the biosensor with each cell in each droplet of the plurality of droplets; and
merging droplets containing the biosensor with a droplet of the plurality of droplets.
10. The method of aspect 8, wherein sorting the droplets comprises using fluorescence-activated droplet sorting.
11. The method of aspect 1, wherein each droplet of the plurality of droplets has a volume ranging from 10 picoliters to about 1000 nanoliters and/or wherein each droplet of the plurality of droplets has a diameter ranging from 5 microns to about 2000 microns.
12. The method of any one of aspects 1-14, wherein the suspension of cells include any one or more of:
a genetically heterogenous or inhomogeneous cell population;
a genetically homogenous cell population;
natural cells;
engineered cells; and
a heterogeneous cell population generated by library approaches.
13. The method of aspect 1, wherein the first cell culture medium and the second cell culture medium are the same.
14. A system for generating and/or identifying cells adapted to replicate in a selected environment, wherein the selected environment comprises an incubating environment, and one or more of a first cell culture medium and a second cell culture, the system comprising:
   a droplet producing device configured to accept a suspension of cells in the first or the second cell culture medium and create droplets, wherein each droplet comprises one or more cells from the first suspension of cells;
   an incubator configured to allow replication of cells within each droplet while in the selected environment;
   a droplet breaking apparatus adapted to accept droplets from the incubator and yield cells in suspension comprising survivor cells as a subset of the cells in the suspension; and
a cell culture apparatus adapted to accept the survivor cells from the droplet breaking apparatus, the cell culture apparatus configured to create cell isolates.
15. The system of aspect 14, wherein the droplet producing device is a two-stream flow-focusing microfluidic device.
16 The system of aspect 15, wherein the two-stream flow-focusing microfluidic device is further configured to accept the cells in suspension created by the droplet breaking apparatus.
17. The system of aspect 14, further comprising a cell dilution apparatus adapted to accept the cells in suspension created by the droplet breaking apparatus, admix the survivor cells with a second cell culture medium to form a second suspension of cells, and provide the second suspension of cells to the droplet producing device.
18. The system of aspect 14, wherein the incubator is configured to allow replication of cells for at least 12 hours.
19. The system of aspect 14, further comprising a biosensor introducing apparatus adapted to accept the droplets from the droplet producing device and insert a biosensor into the droplets.
20 The system of aspect 14, further comprising a sorting apparatus adapted to accept droplets, wherein each droplet contains a cell and a biosensor, to create a collection of selected droplets.
21 A method for identifying cells adapted to replicate in a selected environment, the method comprising use of the system of aspect 14.
22. The system of aspect 14, further comprising an apparatus for determining an amount of desired product produced by each cell isolate.

What is claimed is:

1. A method for generating and/or selecting cells adapted to replicate in a selected environment, the method comprising steps of:
encapsulating cells from a first suspension of cells in a first cell culture medium into a plurality of droplets, each droplet comprising one or more cells from the first suspension of cells;
incubating the plurality of droplets in an incubating environment;
breaking the plurality of droplets to release cells;
resuspending a subset of the released cells in a second cell culture medium to form a second suspension of cells and
re-encapsulating cells from the second suspension of cells to form a plurality of droplets, each droplet comprising one or more cells from the second suspension of cells;
wherein:
the selected environment includes one or more of the first cell culture medium, the second cell culture medium, and the incubating environment; and
the incubating, breaking, resuspending and re-encapsulating steps are repeated to identify cells adapted to replicate in the selected environment.

2. The method of claim 1, wherein the incubating environment comprises any one or more of: percent oxygen (O₂), relative humidity, temperature, and agitation.

3. The method of claim 1, wherein the first culture medium and/or the second cell culture medium comprise:
a feed-stock;
a toxin;
an inhibitor;
a salt; a selected ionic strength;
a selected osmolarity; a biological product or byproduct;
a media component; and
cell debris/waste.

4. The method of claim 1, wherein the incubating, breaking, resuspending and re-encapsulating steps are repeated at least 4 times.

5. The method of claim 1, wherein the incubating, breaking, resuspending and re-encapsulating steps are repeated at least 2 times in a time period of seven days.

6. The method of claim 1, wherein incubating the plurality of droplets is done over a period of time equal to at least 12 hours.

7. The method of claim 1, further comprising collecting the identified cells that are adapted to replicate in the selected environment, and optionally further comprising evaluating the cells adapted to replicate in the selected environment based on ability to produce a desired product.

8. The method of claim 1, further comprising:
introducing a biosensor to the plurality of droplets, wherein the biosensor emits a signal when a desired product produced by the cells is present; and
sorting the plurality of droplets based upon a presence or an absence of the biosensor signal in the droplet.

9. The method of claim 8, wherein introducing the biosensor comprises any of:
injecting the biosensor into each droplet of the plurality of droplets using a pico-injection method;
co-encapsulation of the biosensor with each cell in each droplet of the plurality of droplets; and
merging droplets containing the biosensor with a droplet of the plurality of droplets.

10. The method of claim 8, wherein sorting the droplets comprises using fluorescence-activated droplet sorting.

11. The method of claim 1, wherein each droplet of the plurality of droplets has a volume ranging from 10 picoliters to about 1000 nanoliters and/or wherein each droplet of the plurality of droplets has a diameter ranging from 5 microns to about 2000 microns.

12. The method of claim 1, wherein the first suspension of cells includes any one or more of:
a genetically heterogenous or inhomogeneous cell population;
a genetically homogenous cell population;
natural cells;
engineered cells; and
a heterogeneous cell population generated by library approaches.

13. The method of claim 1, wherein the first cell culture medium and the second cell culture medium are the same.

14. A system for generating and/or identifying cells adapted to replicate in a selected environment, wherein the selected environment comprises an incubating environment, and one or more of a first cell culture medium and a second cell culture medium, the system comprising:
a droplet producing device configured to accept a suspension of cells in the first or second cell culture medium and create droplets, wherein each droplet comprises one or more cells from the suspension of cells;
an incubator configured to allow replication of cells within each droplet while in the selected environment;
a droplet breaking apparatus adapted to accept droplets from the incubator and yield cells in suspension comprising survivor cells as a subset of the cells in suspension; and
a cell culture apparatus adapted to accept the survivor cells from the droplet breaking apparatus, the cell culture apparatus configured to create cell isolates.

15. The system of claim 14, wherein the droplet producing device is a two-stream flow-focusing microfluidic device.

16. The system of claim 15, wherein the two-stream flow-focusing microfluidic device is further configured to accept the cells in suspension created by the droplet breaking apparatus.

17. The system of claim 14, further comprising a cell dilution apparatus adapted to accept the cells in suspension created by the droplet breaking apparatus, admix the survivor cells with a second cell culture medium to form a second suspension of cells, and provide the second suspension of cells to the droplet producing device.

18. The system of claim 14, further comprising a biosensor introducing apparatus adapted to accept the droplets from the droplet producing device and insert a biosensor into the droplets.

19. The system of claim 14, further comprising a sorting apparatus adapted to accept droplets, wherein each droplet contains a cell and a biosensor, to create a collection of selected droplets.

20. A method for identifying cells adapted to replicate in a selected environment, the method comprising use of the system of claim 14.
